(19) United States
(12) Patent Application Publication
(10) Pub. No.: US 2026/0076648 A1
(43) Pub. Date: Mar. 19, 2026

(54) OPERATION SUPPORT SYSTEM FOR ULTRASOUND PROBE AND OPERATION SUPPORT PROGRAM FOR ULTRASOUND PROBE

(71) Applicant: FUJIFILM Corporation, Tokyo (JP)

(72) Inventor: Shuichi DOKKO, Tokyo (JP)

(73) Assignee: FUJIFILM Corporation, Tokyo (JP)

(21) Appl. No.: 19/329,493

(22) Filed: Sep. 15, 2025

(30) Foreign Application Priority Data
Sep. 18, 2024 (JP) 2024-161530

(51) Int. Cl. A61B 8/00

(52) U.S. Cl. CPC A61B 8/4263; A61B 8/4218

(57) ABSTRACT

A probe detection mark is attached to an ultrasound probe, and a target detection mark is attached to a treatment target. A pre-treatment relative relationship calculation unit calculates a pre-treatment relative relationship between the ultrasound probe and the treatment target at a pre-treatment ultrasound transmission and reception time, based on a pre-treatment camera image including an image of the probe detection mark and an image of the target detection mark, which are formed before a treatment on the treatment target. A current relative relationship calculation unit calculates a current relative relationship between the ultrasound probe and the treatment target based on a current camera image including an image of the probe detection mark and an image of the target detection mark, which are formed at a current time. A probe control support unit executes control of bringing the current relative relationship closer to the pre-treatment relative relationship.

This application claims priority to Japanese Patent Application No. 2024-161530 filed on Sep. 18, 2024 which is incorporated herein by reference in its entirety including the specification, claims, drawings, and abstract.

### BACKGROUND OF THE INVENTION

### 1. Field of the Invention

The present specification discloses improvements in an operation support system for an ultrasound probe and an operation support program for an ultrasound probe.

### 2. Description of the Related Art

An ultrasound diagnostic apparatus that transmits ultrasound from an ultrasound probe toward a subject, receives a reflected wave from the subject in the ultrasound probe, and performs various types of processing such as formation of an ultrasound tomographic image representing a cross section in the subject, formation of a Doppler image representing a velocity of a tissue (blood or the like) in the subject, and various types of measurement based on a reception signal formed from the reflected wave has been known.

In the related art, in order for an examiner to appropriately perform desired processing with the ultrasound diagnostic apparatus, it is necessary to acquire an appropriate reception signal. In order to acquire an appropriate reception signal, it is necessary to appropriately set a position and an orientation of the ultrasound probe. Therefore, in the related art, various techniques for supporting an appropriate position and an appropriate orientation of the ultrasound probe have been proposed.

For example, JP2013-255658A discloses an ultrasound diagnostic apparatus comprising: a region-of-interest (ROI) mark setting unit that sets a region of interest designated by an examiner on an ultrasound image; a camera that images a subject to which a subject AR mark is attached and an ultrasound probe to which an ultrasound probe AR mark is attached, to acquire a camera image; a non-subject coordinate conversion unit that calculates a body surface curve approximating a body surface of the subject based on the subject AR mark in the camera image; an ultrasound probe coordinate conversion unit that calculates 3D coordinate information of the ultrasound probe based on the ultrasound probe AR mark in the camera image; a 3D processor that calculates a position of the region of interest on the body surface of the subject based on the region of interest, the body surface curve of the subject, and the 3D coordinate information of the ultrasound probe; and an ROI/camera image combining unit that displays an ROI projection mark indicating the position of the region of interest on the camera image in a superimposed manner.

In addition, JP4999012B discloses a robot surgical system comprising: a first robot arm that holds an ultrasound probe; a second robot arm that holds a surgical instrument; and a master input device for remotely operating a plurality of robot arms, in which the robot surgical system is operable in a first mode in which the second robot arm is fixed at a predetermined position and the first robot arm is moved in accordance with the operation of the master input device, or a second mode in which the second robot arm is moved in accordance with the operation of the master input device and the first robot arm is moved in accordance with a user command.

### SUMMARY OF THE INVENTION

In a case in which a treatment (for example, surgery) is performed on a treatment target (for example, an organ) of the subject, the ultrasound is transmitted to and received from the treatment target before the treatment and after the start of the treatment (that is, during the treatment or after the treatment). In such a case, it may be desired to make the position and the orientation of the ultrasound probe, more specifically, a relative position and a relative orientation of the ultrasound probe with respect to the treatment target (may be referred to as a “relative position and orientation relationship” in the present specification), as similar as possible between before the treatment and after the start of the treatment.

For example, before the treatment, the ultrasound is transmitted to and received from the treatment target of the subject with the ultrasound probe to form the ultrasound tomographic image. A doctor or the like who performs the treatment checks the ultrasound tomographic image to check the treatment target before the treatment. Since the ultrasound probe obstructs the treatment on the treatment target, the ultrasound probe is moved before the treatment is started. After the start of the treatment, for the purpose of checking progress or a result of the treatment, the ultrasound probe transmits and receives the ultrasound to and from the treatment target again to form the ultrasound tomographic image. Here, in order to suitably check the result of the treatment, it is preferable that the ultrasound tomographic image formed before the treatment and the ultrasound tomographic image formed after the start of the treatment represent the same cross section as closely as possible. Stated another way, it is preferable that the relative position and orientation relationship between the ultrasound probe and the treatment target is as similar as possible between before the treatment on the subject and after the start of the treatment.

An object of an operation support system for an ultrasound probe disclosed in the present specification is to reduce a difference in a relative position and orientation relationship between the ultrasound probe and a treatment target between before a treatment on a subject and after a start of the treatment.

An operation support system for an ultrasound probe disclosed in the present specification comprises: a pre-treatment relative relationship calculation unit that calculates, based on a pre-treatment camera image obtained by imaging a probe detection mark attached to an ultrasound probe and a target detection mark attached to a treatment target with a camera before a treatment on a subject, a pre-treatment relative relationship that is a relative position and orientation relationship between the ultrasound probe and the treatment target in a case in which ultrasound is transmitted to and received from the subject for forming an ultrasound image before the treatment on the subject; a current relative relationship calculation unit that calculates, based on a current camera image obtained by imaging the probe detection mark and the target detection mark with the camera at a current time after a start of the treatment on the subject, a current relative relationship that is a relative position and orientation relationship between the ultrasound probe and the treatment target at the current time; and a probe control support unit that executes control of bringing the current relative relationship closer to the pre-treatment relative relationship.

The probe control support unit may bring the current relative relationship closer to the pre-treatment relative relationship by changing a position or an orientation of a robot arm that holds the ultrasound probe.

The probe control support unit may notify an operator who operates the ultrasound probe of a position and an orientation of the ultrasound probe for achieving the current relative relationship.

The probe control support unit may display, on a display unit, a camera image that is obtained by imaging the ultrasound probe and the treatment target with the camera and that shows the position and the orientation of the ultrasound probe for achieving the current relative relationship.

The pre-treatment relative relationship calculation unit may convert, in accordance with an arm-probe position and orientation relationship that is a fixed position and orientation relationship between a robot arm and the ultrasound probe held by the robot arm, position and orientation information indicating a position and an orientation of the ultrasound probe, in a camera coordinate system, specified based on an image of the probe detection mark included in the pre-treatment camera image into position and orientation information, in a robot coordinate system, recognized by a robot control device that controls the robot arm, convert, in accordance with the arm-probe position and orientation relationship, position and orientation information indicating a position and an orientation of the treatment target, in the camera coordinate system, specified based on an image of the target detection mark included in the pre-treatment camera image into position and orientation information in the robot coordinate system, and calculate the pre-treatment relative relationship in the robot coordinate system, and the current relative relationship calculation unit may convert, in accordance with the arm-probe position and orientation relationship, position and orientation information indicating a position and an orientation of the ultrasound probe, in the camera coordinate system, specified based on an image of the probe detection mark included in the current camera image into position and orientation information in the robot coordinate system, convert, in accordance with the arm-probe position and orientation relationship, position and orientation information indicating a position and an orientation of the treatment target, in the camera coordinate system, specified based on an image of the target detection mark included in the current camera image into position and orientation information in the robot coordinate system, and calculate the current relative relationship in the robot coordinate system.

The pre-treatment relative relationship calculation unit may convert, in accordance with an arm-camera position and orientation relationship that is a fixed position and orientation relationship between a robot arm and the camera held by the robot arm, position and orientation information indicating a position and an orientation of the ultrasound probe, in a camera coordinate system, specified based on an image of the probe detection mark included in the pre-treatment camera image into position and orientation information, in a robot coordinate system, recognized by a robot control device that controls the robot arm, convert, in accordance with the arm-camera position and orientation relationship, position and orientation information indicating a position and an orientation of the treatment target, in the camera coordinate system, specified based on an image of the target detection mark included in the pre-treatment camera image into position and orientation information in the robot coordinate system, and calculate the pre-treatment relative relationship in the robot coordinate system, and the current relative relationship calculation unit may convert, in accordance with the arm-camera position and orientation relationship, position and orientation information indicating a position and an orientation of the ultrasound probe, in the camera coordinate system, specified based on an image of the probe detection mark included in the current camera image into position and orientation information in the robot coordinate system, convert, in accordance with the arm-camera position and orientation relationship, position and orientation information indicating a position and an orientation of the treatment target, in the camera coordinate system, specified based on an image of the target detection mark included in the current camera image into position and orientation information in the robot coordinate system, and calculate the current relative relationship in the robot coordinate system.

The pre-treatment camera image and the current camera image may be obtained by imaging the probe detection mark, the target detection mark, and an arm detection mark attached to a robot arm with the camera, the pre-treatment relative relationship calculation unit may convert, in accordance with an inter-coordinate system relationship indicating a relationship between camera coordinates and robot coordinates and obtained based on a position and an orientation of the robot arm, in a camera coordinate system, specified based on an image of the arm detection mark included in the pre-treatment camera image and a position and an orientation of the robot arm, in a robot coordinate system, recognized by a robot control device that controls the robot arm, position and orientation information indicating a position and an orientation of the ultrasound probe, in the camera coordinate system, specified based on an image of the probe detection mark included in the pre-treatment camera image into position and orientation information in the robot coordinate system, convert, in accordance with the inter-coordinate system relationship, position and orientation information indicating a position and an orientation of the treatment target, in the camera coordinate system, specified based on an image of the target detection mark included in the pre-treatment camera image into position and orientation information in the robot coordinate system, and calculate the pre-treatment relative relationship in the robot coordinate system, and the current relative relationship calculation unit may convert, in accordance with the inter-coordinate system relationship, position and orientation information indicating a position and an orientation of the ultrasound probe, in the camera coordinate system, specified based on an image of the probe detection mark included in the current camera image into position and orientation information in the robot coordinate system, convert, in accordance with the inter-coordinate system relationship, position and orientation information indicating a position and an orientation of the treatment target, in the camera coordinate system, specified based on an image of the target detection mark included in the current camera image into position and orientation information in the robot coordinate system, and calculate the current relative relationship in the robot coordinate system.

In addition, an operation support program for an ultrasound probe disclosed in the present specification causes a computer to function as: a pre-treatment relative relationship calculation unit that calculates, based on a pre-treatment camera image obtained by imaging a probe detection mark attached to an ultrasound probe and a target detection mark attached to a treatment target with a camera before a treatment on a subject, a pre-treatment relative relationship that is a relative position and orientation relationship between the ultrasound probe and the treatment target in a case in which ultrasound is transmitted to and received from the subject for forming an ultrasound image before the treatment on the subject; a current relative relationship calculation unit that calculates, based on a current camera image obtained by imaging the probe detection mark and the target detection mark with the camera at a current time after a start of the treatment on the subject, a current relative relationship that is a relative position and orientation relationship between the ultrasound probe and the treatment target at the current time; and a probe control support unit that executes control of bringing the current relative relationship closer to the pre-treatment relative relationship.

With the operation support system for the ultrasound probe disclosed in the present specification, it is possible to reduce the difference in the relative position and orientation relationship between the ultrasound probe and the treatment target between before the treatment on the subject and after the start of the treatment.

### BRIEF DESCRIPTION OF THE DRAWINGS

FIG. 1 is a schematic configuration diagram of an operation support system for an ultrasound probe according to a first embodiment.

FIG. 2 is a conceptual diagram showing an endoscopic camera, an ultrasound probe, and a treatment target in the first embodiment.

FIG. 3 is a schematic configuration diagram of an ultrasound diagnostic apparatus.

FIG. 4 is a diagram showing an example of a guide screen of the ultrasound probe.

FIG. 5 is a flowchart showing a flow of processing before a treatment of the ultrasound diagnostic apparatus according to the first embodiment.

FIG. 6 is a flowchart showing a flow of processing after a start of the treatment of the ultrasound diagnostic apparatus according to the first embodiment.

FIG. 7 is a schematic configuration diagram of an operation support system for an ultrasound probe according to a second embodiment.

FIG. 8 is a conceptual diagram showing an extracorporeal camera, the ultrasound probe, and a subject in the second embodiment.

FIG. 9 is a schematic configuration diagram of an operation support system for an ultrasound probe according to a third embodiment.

FIG. 10 is a flowchart showing a flow of processing before a treatment of the ultrasound diagnostic apparatus according to the third embodiment.

FIG. 11 is a flowchart showing a flow of processing after a start of the treatment of the ultrasound diagnostic apparatus according to the third embodiment.

FIG. 12 is a schematic configuration diagram of an operation support system for an ultrasound probe according to a fourth embodiment.

FIG. 13 is a flowchart showing a flow of processing before a treatment of the ultrasound diagnostic apparatus according to the fourth embodiment.

FIG. 14 is a flowchart showing a flow of processing after a start of the treatment of the ultrasound diagnostic apparatus according to the fourth embodiment.

### DESCRIPTION OF THE PREFERRED EMBODIMENTS

### First Embodiment

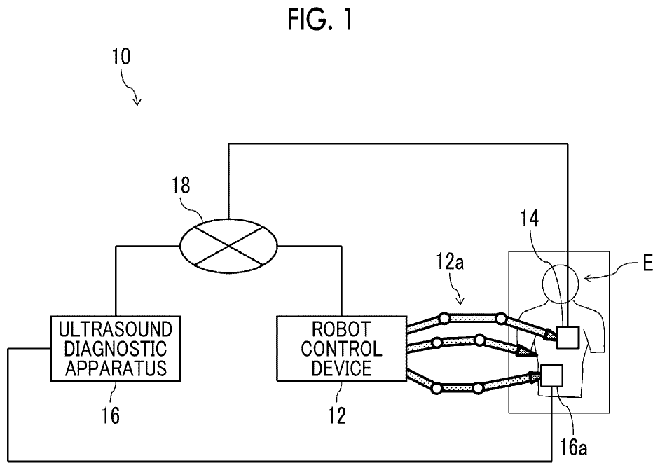

FIG. 1 is a schematic configuration diagram of an operation support system 10 for an ultrasound probe according to a first embodiment. The operation support system 10 for the ultrasound probe includes a robot control device 12 including a plurality of robot arms 12a, an endoscope 14, and an ultrasound diagnostic apparatus 16 including an ultrasound probe 16a. The robot control device 12, the endoscope 14, and the ultrasound diagnostic apparatus 16 are connected to each other via a communication line 18 such as a wide area network (WAN) or a local area network (LAN) so as to be able to communicate with each other.

The robot control device 12 includes, in addition to the plurality of robot arms 12a, a processor composed of a central processing unit (CPU) and the like, a communication interface composed of a network adapter and the like, and an input interface composed of a surgeon console and the like. The processor of the robot control device 12 controls the robot arm 12a in accordance with an instruction from an operator such as a doctor. The operator may directly input the instruction to the robot control device 12 using the input interface of the robot control device 12, or may remotely input the instruction via the communication line 18.

The robot arm 12a holds a treatment tool (for example, scissors or forceps) for performing a treatment. Stated another way, the robot control device 12 controls the treatment (for example, surgery) on a subject E using the treatment tool.

In the first embodiment, the robot arm 12a holds the endoscope 14 and the ultrasound probe 16a. Stated another way, in the first embodiment, the robot control device 12 controls a position and an orientation of the endoscope 14 and a position and an orientation of the ultrasound probe 16a.

The endoscope 14 includes, in addition to a lens and an image sensor, a processor composed of a CPU and the like, a communication interface composed of a network adapter and the like, and the like. The endoscope 14 is a camera that images an inside of a body cavity of the subject E. An endoscopic image is formed by the image sensor of the endoscope 14, and the endoscopic image is transmitted to the ultrasound diagnostic apparatus 16 via the communication interface of the endoscope 14. The endoscope 14 may be directly connected to the ultrasound diagnostic apparatus 16 through a cable or the like without going through the communication line 18, and the endoscopic image may be directly transmitted to the ultrasound diagnostic apparatus 16.

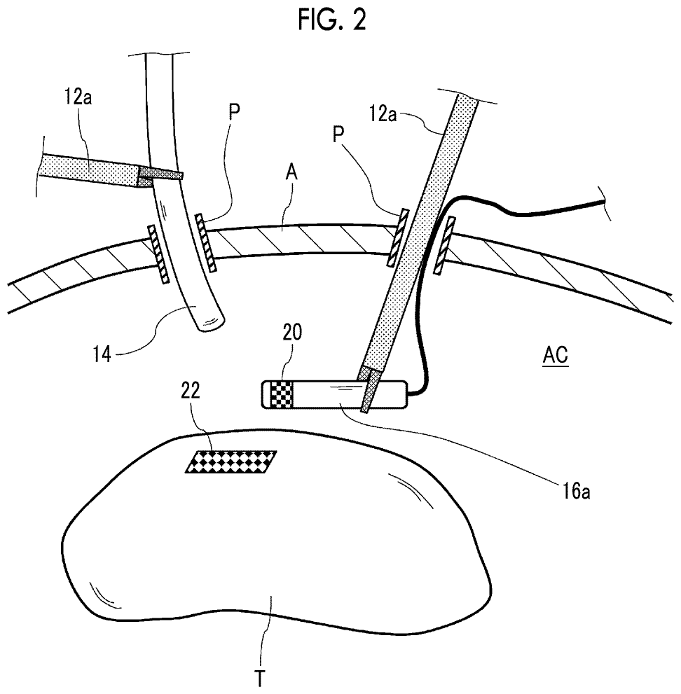

FIG. 2 is a conceptual diagram showing the endoscope 14, the ultrasound probe 16a, and a treatment target T (in the example of FIG. 2, an organ). In the first embodiment, a case is assumed in which laparoscopic surgery is performed on the subject E. In the laparoscopic surgery, a small hole is opened in an abdomen A of the subject E, and a sleeve (port) P is attached to the hole. An abdominal cavity AC is inflated with gas, and the endoscope 14, the ultrasound probe 16a, and the treatment tool (not shown) are inserted into the abdominal cavity AC from the sleeve P. In the first embodiment, the ultrasound probe 16a is a drop-in type probe that is inserted into the body cavity (in the example of FIG. 2, the abdominal cavity). As described above, in the first embodiment, the endoscope 14 and the ultrasound probe 16a are held by the robot arm 12a, and the robot control device 12 controls the position and the orientation of the endoscope 14 and the position and the orientation of the ultrasound probe 16a.

A probe detection mark 20 is attached to the ultrasound probe 16a. The probe detection mark 20 is a mark for detecting the position and the orientation of the ultrasound probe 16a. The position and the orientation of the ultrasound probe 16a can be detected by imaging the probe detection mark 20 with the camera to acquire a camera image (in the first embodiment, an endoscopic image) and analyzing an image of the probe detection mark 20 included in the camera image. Examples of the probe detection mark 20 include an augmented reality (AR) marker.

In addition, a target detection mark 22 is attached to the treatment target T. The target detection mark 22 is a mark for detecting the position and the orientation of the treatment target T. Accordingly, the position and the orientation of the target detection mark 22 can be detected by imaging the target detection mark 22 with the camera to acquire the camera image and analyzing an image of the target detection mark 22 included in the camera image. Examples of the target detection mark 22 include an AR marker. The target detection mark 22 has a pattern different from a pattern of the probe detection mark 20.

Here, a procedure of the treatment on the treatment target T will be described. First, before the treatment on the treatment target T, the ultrasound is transmitted to and received from the treatment target T by the ultrasound probe 16a inserted into the abdominal cavity AC. As a result, an ultrasound tomographic image is formed in the ultrasound diagnostic apparatus 16. In the present specification, the ultrasound tomographic image will be referred to as a pre-treatment ultrasound tomographic image. The operator can check a cross section of the treatment target T using the pre-treatment ultrasound tomographic image. In addition, at a time when the ultrasound is transmitted to and received from the treatment target T for forming the pre-treatment ultrasound tomographic image (in the present specification, referred to as a pre-treatment ultrasound transmission and reception time), the endoscope 14 images the probe detection mark 20 and the target detection mark 22 to form the camera image (in the first embodiment, the endoscopic image). In the present specification, the camera image will be referred to as a pre-treatment camera image. The pre-treatment camera image includes the image of the probe detection mark 20 and the image of the target detection mark 22. In particular, the image of the probe detection mark 20 is information indicating the position and the orientation of the ultrasound probe 16a at the pre-treatment ultrasound transmission and reception time, and the image of the target detection mark 22 is information indicating the position and the orientation of the treatment target T (specifically, a surface thereof) at the pre-treatment ultrasound transmission and reception time. The pre-treatment camera image is transmitted from the endoscope 14 to the ultrasound diagnostic apparatus 16.

Thereafter, the treatment on the treatment target T is started by the operator. Since the ultrasound probe 16a obstructs the treatment on the treatment target T, the ultrasound probe 16a is temporarily brought out of the abdominal cavity AC. At least the position and the orientation of the ultrasound probe 16a are changed from the position and the orientation at the pre-treatment ultrasound transmission and reception time.

After the start of the treatment, the ultrasound probe 16a is inserted into the abdominal cavity AC again, and the transmission and reception of the ultrasound to and from the treatment target T are attempted again by the ultrasound probe 16a in order to check progress or a result of the treatment. In the present specification, the ultrasound tomographic image formed after the start of the treatment (that is, a current time) will be referred to as a current ultrasound tomographic image. Here, in order to suitably compare the pre-treatment ultrasound tomographic image with the current ultrasound tomographic image, the position and the orientation of the ultrasound probe 16a at the current time may be made as similar as possible to the position and the orientation of the ultrasound probe 16a at the pre-treatment ultrasound transmission and reception time.

For this purpose, at the current time, the endoscope 14 images the probe detection mark 20 and the target detection mark 22 to form the camera image (in the first embodiment, the endoscopic image). In the present specification, the camera image will be referred to as a current camera image. The current camera image also includes the image of the probe detection mark 20 and the image of the target detection mark 22. In particular, the image of the probe detection mark 20 is information indicating the position and the orientation of the ultrasound probe 16a at the current time, and the image of the target detection mark 22 is information indicating the position and the orientation of the treatment target T (specifically, the surface thereof) at the current time. The current camera image may be a video image, and the current camera image including the image of the probe detection mark 20 and the image of the target detection mark 22 at the current time is always formed. The current camera image is transmitted from the endoscope 14 to the ultrasound diagnostic apparatus 16.

As will be described in detail below, in the ultrasound diagnostic apparatus 16, the pre-treatment camera image and the current camera image are analyzed to assist in making the position and the orientation of the ultrasound probe 16a after the start of the treatment (that is, at the current time) as similar as possible to the position and the orientation of the ultrasound probe 16a at the pre-treatment ultrasound transmission and reception time.

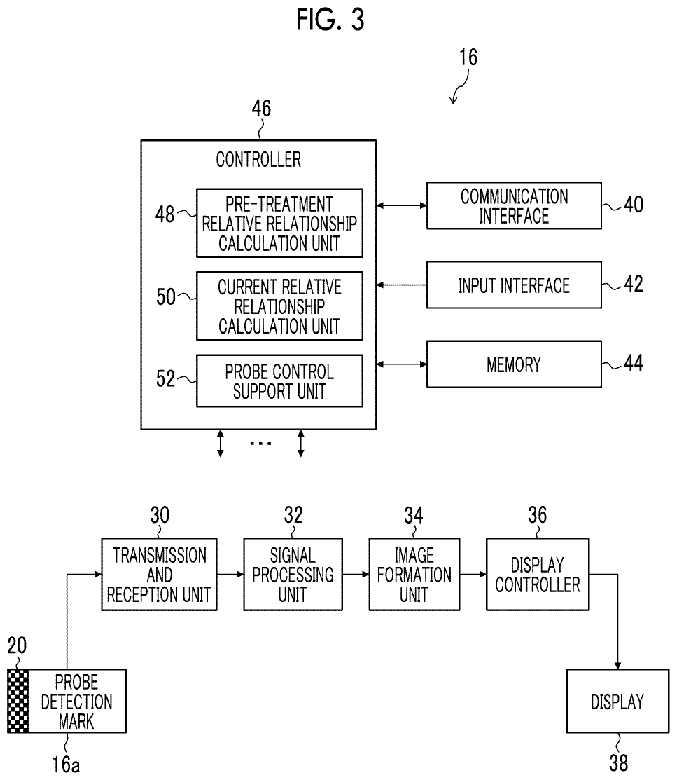

FIG. 3 is a schematic configuration diagram of the ultrasound diagnostic apparatus 16. The ultrasound diagnostic apparatus 16 is a medical apparatus installed in medical institutions, such as a hospital.

The ultrasound probe 16a is a device that transmits and receives the ultrasound to and from the treatment target T of the subject E. The ultrasound probe 16a has a vibration element array consisting of a plurality of vibration elements that transmit and receive the ultrasound to and from the treatment target T. In the ultrasound probe 16a, the vibration element array is formed of the plurality of vibration elements arranged in one row. A transmission signal is supplied to each vibration element from a transmission and reception unit 30 described later, so that each vibration element generates the ultrasound.

In this way, the probe detection mark 20 is attached to the ultrasound probe 16a.

The transmission and reception unit 30 transmits the transmission signal to the ultrasound probe 16a (specifically, each vibration element of the vibration element array) under control of a controller 46 described later. In addition, the transmission and reception unit 30 receives the reception signal from each vibration element that has received the reflected wave reflected by the treatment target T. The transmission and reception unit 30 has an adder and a plurality of delay elements corresponding to the respective vibration elements, and performs phase addition processing of aligning and adding phases of reception signals from the respective vibration elements by the adder and the plurality of delay elements. As a result, a reception beam signal is formed in which information indicating a signal intensity of the reflected wave reflected by the treatment target T is arranged in a depth direction of the treatment target T.

The signal processing unit 32 executes various types of signal processing including filter processing of applying a bandpass filter, detection processing, and the like, on the reception beam signal from the transmission and reception unit 30.

The image formation unit 34 forms the ultrasound tomographic image (B-mode image) representing the cross section (particularly, an ultrasound transmission/reception surface) of the treatment target T based on the reception beam signal on which the signal processing has been performed by the signal processing unit 32.

The display controller 36 performs control to display various images including the ultrasound tomographic image formed by the image formation unit 34 on a display 38.

The display 38 as a display unit is, for example, a display device composed of a liquid-crystal display, an organic electroluminescence (EL), or the like.

Although not shown in FIG. 3, the ultrasound diagnostic apparatus 16 may include a Doppler signal formation unit that forms a Doppler signal indicating a movement of the treatment target T based on the reception signal obtained by the transmission and reception unit 30, a color Doppler image formation unit that forms a color Doppler image based on the Doppler signal, a measurement unit that performs various measurements based on the reception signal obtained by the transmission and reception unit 30, and the like.

The transmission and reception unit 30, the signal processing unit 32, the image formation unit 34, and the display controller 36, which are included in the ultrasound diagnostic apparatus 16, and the Doppler signal formation unit, the color Doppler image formation unit, and the measurement unit, which are described above, are configured by a processor. The processor includes at least one of a general-purpose processing device (for example, a CPU or the like) or a dedicated processing device (for example, a graphics processing unit (GPU), an application-specific integrated circuit (ASIC), a field-programmable gate array (FPGA), or a programmable logic device). The processor may be configured not by a single processing device, but by cooperation among a plurality of processing devices that are physically separated from each other. Additionally, each of the above-described units may be implemented through cooperation of hardware, such as a processor, and software.

A communication interface 40 is composed of, for example, a network adapter and the like. The communication interface 40 exhibits a function of communicating with other devices (particularly, the robot control device 12 and the endoscope 14) via the communication line 18. In particular, in the first embodiment, the communication interface 40 receives the endoscopic image from the endoscope 14, receives position and orientation information indicating the position and the orientation of each robot arm 12a from the robot control device 12, and transmits a control signal for controlling the robot arm 12a (that is, the ultrasound probe 16a) to the robot control device 12.

An input interface 42 is composed of, for example, a button, a trackball, and a touch panel. The input interface 42 is used to input the instruction of the operator, who uses the ultrasound diagnostic apparatus 16, to the ultrasound diagnostic apparatus 16.

A memory 44 includes a hard disk drive (HDD), a solid-state drive (SSD), an embedded MultiMediaCard (eMMC), a read-only memory (ROM), or a random-access memory (RAM). An operation support program for the ultrasound probe for operating each unit of the ultrasound diagnostic apparatus 16 is stored in the memory 44. The operation support program for the ultrasound probe can also be stored in, for example, a computer-readable non-transitory storage medium such as a Universal Serial Bus (USB) memory or a CD-ROM. The ultrasound diagnostic apparatus 16 can read the operation support program for the ultrasound probe from such a storage medium and execute the read operation support program. Since the ultrasound diagnostic apparatus 16 reads an operation support program for the ultrasound probe to exhibit functions described later, it can be said that the ultrasound diagnostic apparatus 16 is a computer program product.

A controller 46 includes at least one of a general-purpose processor (such as a CPU, for example) or a dedicated processor (for example, a GPU, an ASIC, an FPGA, or a programmable logic device). The controller 46 may be configured not by a single processing device, but by cooperation among a plurality of processing devices that are physically separated from each other. The controller 46 controls each unit of the ultrasound diagnostic apparatus 16. In addition, as shown in FIG. 3, the controller 46 exhibits functions as a pre-treatment relative relationship calculation unit 48, a current relative relationship calculation unit 50, and a probe control support unit 52 in accordance with the operation support program for the ultrasound probe stored in the memory 44.

The pre-treatment relative relationship calculation unit 48 calculates a relative positional relationship (in the present specification, referred to as a pre-treatment relative relationship) between the ultrasound probe 16a and the treatment target T at the pre-treatment ultrasound transmission and reception time based on the pre-treatment camera image formed by the endoscope 14 before the treatment on the treatment target T.

As described above, the pre-treatment camera image includes the image of the probe detection mark 20 indicating the position and the orientation of the ultrasound probe 16a at the pre-treatment ultrasound transmission and reception time and the image of the target detection mark 22 indicating the position and the orientation of the treatment target T at the pre-treatment ultrasound transmission and reception time. The pre-treatment relative relationship calculation unit 48 analyzes the image of the probe detection mark 20 in the pre-treatment camera image to detect the position and the orientation of the ultrasound probe 16a, in a camera coordinate system of the endoscope 14, at the pre-treatment ultrasound transmission and reception time. Similarly, the pre-treatment relative relationship calculation unit 48 analyzes the image of the target detection mark 22 in the pre-treatment camera image to detect the position and the orientation of the treatment target T (the surface thereof, the same applies hereinafter), in the camera coordinate system of the endoscope 14, at the pre-treatment ultrasound transmission and reception time. Since a known method can be used as the method of detecting the position and the orientation of the ultrasound probe 16a or the treatment target T in the camera coordinate system from the image of the probe detection mark 20 or the image of the target detection mark 22 included in the pre-treatment camera image, the detailed description thereof will be omitted here.

Next, the pre-treatment relative relationship calculation unit 48 converts position and orientation information indicating the position and the orientation of the ultrasound probe 16a and the position and the orientation of the treatment target T in the camera coordinate system (hereinafter, simply referred to as position and orientation information of the ultrasound probe 16a and position and orientation information of the treatment target T, respectively) into position and orientation information in a robot coordinate system. The robot coordinate system is a coordinate system that is recognized by the robot control device 12 and is used to represent the position and the orientation of the robot arm 12a.

In the first embodiment, since the ultrasound probe 16a is held by the robot arm 12a, the position and orientation relationship between the ultrasound probe 16a and the robot arm 12a is fixed (not changed). In the present specification, a fixed position and orientation relationship between the ultrasound probe 16a and the robot arm 12a will be referred to as an arm-probe position and orientation relationship. The arm-probe position and orientation relationship can be represented by, for example, a vector in the robot coordinate system. In the first embodiment, the pre-treatment relative relationship calculation unit 48 converts, in accordance with the arm-probe position and orientation relationship, the position and orientation information of the ultrasound probe 16a in the camera coordinate system into the position and orientation information in the robot coordinate system.

Specifically, the pre-treatment relative relationship calculation unit 48 acquires the position and orientation information of the robot arm 12a that holds the ultrasound probe 16a in the robot coordinate system and the information indicating the arm-probe position and orientation relationship from the robot control device 12. The information indicating the arm-probe position and orientation relationship may be input to the robot control device 12 by, for example, the operator who causes the robot arm 12a to hold the ultrasound probe 16a. The pre-treatment relative relationship calculation unit 48 can obtain the position and orientation information of the ultrasound probe 16a in the robot coordinate system by using the position and orientation information of the robot arm 12a in the robot coordinate system and the arm-probe position and orientation relationship. In the present specification, the position and orientation information of the ultrasound probe 16a at the pre-treatment ultrasound transmission and reception time will be referred to as pre-treatment probe position and orientation information.

In addition, in the first embodiment, the pre-treatment relative relationship calculation unit 48 converts, in accordance with the arm-probe position and orientation relationship, the position and orientation information of the treatment target T in the camera coordinate system into the position and orientation information in the robot coordinate system. As described above, the pre-treatment relative relationship calculation unit 48 can obtain the position and orientation information of the ultrasound probe 16a in the camera coordinate system and the position and orientation information of the ultrasound probe 16a in the robot coordinate system. By aligning the respective axial directions of the camera coordinate system and the robot coordinate system, a conversion vector from the camera coordinate system to the robot coordinate system can be obtained based on the position and orientation information of the ultrasound probe 16a in the camera coordinate system and the robot coordinate system. The pre-treatment relative relationship calculation unit 48 can convert the position and orientation information of the treatment target T in the camera coordinate system into the position and orientation information in the robot coordinate system by using the conversion vector. In the present specification, the position and orientation information of the treatment target T at the pre-treatment ultrasound transmission and reception time will be referred to as pre-treatment target position and orientation information.

The pre-treatment relative relationship calculation unit 48 calculates a pre-treatment relative relationship in the robot coordinate system based on the pre-treatment probe position and orientation information and the pre-treatment target position and orientation information obtained as described above. The pre-treatment relative relationship can be represented by a difference between the pre-treatment probe position and orientation information and the pre-treatment target position and orientation information. The pre-treatment relative relationship calculation unit 48 stores the calculated pre-treatment relative relationship, the pre-treatment probe position and orientation information, and the pre-treatment target position and orientation information in the memory 44.

The current relative relationship calculation unit 50 calculates a relative positional relationship (in the present specification, referred to as a current relative relationship) between the ultrasound probe 16a and the treatment target T at the current time based on the current camera image formed by the endoscope 14 after the start of the treatment on the treatment target T (that is, the current time).

As described above, the current camera image includes the image of the probe detection mark 20 indicating the position and the orientation of the ultrasound probe 16a at the current time and the image of the target detection mark 22 indicating the position and the orientation of the treatment target T at the current time. The current relative relationship calculation unit 50 detects the position and the orientation of the ultrasound probe 16a at the current time in the camera coordinate system of the endoscope 14 by analyzing the image of the probe detection mark 20 in the current camera image, similarly to the pre-treatment relative relationship calculation unit 48. Similarly, the current relative relationship calculation unit 50 detects the position and the orientation of the treatment target T at the current time in the camera coordinate system of the endoscope 14 by analyzing the image of the target detection mark 22 in the current camera image.

Next, the current relative relationship calculation unit 50 converts the position and orientation information of the ultrasound probe 16a and the treatment target T in the camera coordinate system into the position and orientation information in the robot coordinate system.

In the first embodiment, the current relative relationship calculation unit 50 converts, in accordance with the arm-probe position and orientation relationship, the position and orientation information of the ultrasound probe 16a in the camera coordinate system into the position and orientation information in the robot coordinate system by the same method as the pre-treatment relative relationship calculation unit 48. In the present specification, the position and orientation information of the ultrasound probe 16a at the current time will be referred to as current probe position and orientation information.

In addition, in the first embodiment, the current relative relationship calculation unit 50 converts, in accordance with the arm-probe position and orientation relationship, the position and orientation information of the treatment target T in the camera coordinate system into the position and orientation information in the robot coordinate system by the same method as the pre-treatment relative relationship calculation unit 48. In the present specification, the position and orientation information of the treatment target T at the current time will be referred to as current target position and orientation information.

The current relative relationship calculation unit 50 calculates a current relative relationship based on the current probe position and orientation information and the current target position and orientation information in the robot coordinate system obtained as described above. The current relative relationship can be represented by a difference between the current probe position and orientation information and the current target position and orientation information.

The probe control support unit 52 executes control of bringing the current relative relationship calculated by the current relative relationship calculation unit 50 closer to the pre-treatment relative relationship calculated by the pre-treatment relative relationship calculation unit 48 and stored in the memory 44.

In the first embodiment, since the robot arm 12a holds the ultrasound probe 16a, the probe control support unit 52 transmits a control signal for changing the position or the orientation of the robot arm 12a that holds the ultrasound probe 16a to the robot control device 12. Specifically, the probe control support unit 52 calculates the position and the orientation of the ultrasound probe 16a (the position and the orientation of the target ultrasound probe 16a) with respect to the position and the orientation of the treatment target T indicated by the current target position and orientation information for achieving the current relative relationship and the pre-treatment relative relationship that are the same as each other, and controls the robot arm 12a that holds the ultrasound probe 16a such that a desired position and a desired orientation of the ultrasound probe 16a are obtained from the position and the orientation of the ultrasound probe 16a indicated by the current probe position and orientation information.

In addition, as described above, the robot arm 12a that holds the ultrasound probe 16a can be controlled by the operator. Therefore, the probe control support unit 52 may notify the operator of the position and the orientation of the ultrasound probe 16a for achieving the current relative relationship. Various methods can be employed as the notification method, and, for example, the probe control support unit 52 may display the endoscopic image that is the camera image that is obtained by imaging the ultrasound probe 16a and the treatment target T with the endoscope 14 and that shows the position and the orientation of the ultrasound probe 16a for achieving the current relative relationship, on the display 38 as a guide screen of the ultrasound probe 16a.

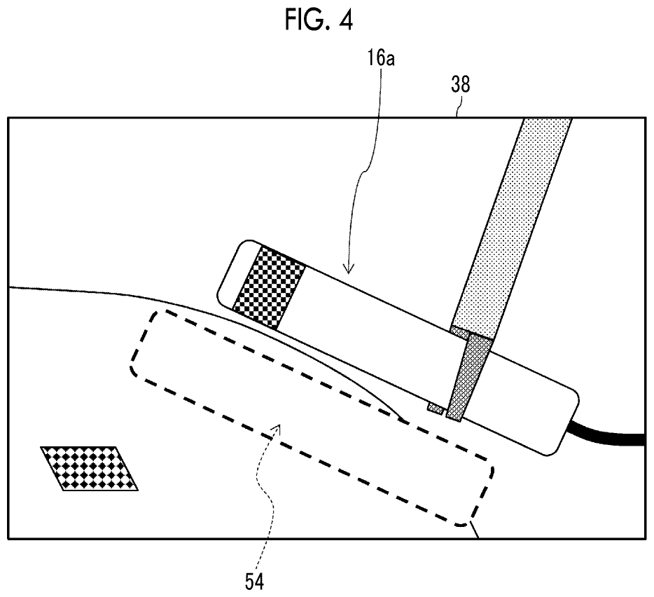

FIG. 4 is a diagram showing an example of a guide screen of the ultrasound probe 16a. As shown in FIG. 4, the probe control support unit 52 may display, for example, a guide 54 indicating the position and the orientation of the ultrasound probe 16a for achieving the current relative relationship, in the endoscopic image. In the example of FIG. 4, the guide 54 indicates an outer shape of the image of the ultrasound probe 16a for achieving the current relative relationship. The operator can match the current relative relationship with the pre-treatment relative relationship by changing the position and the orientation of the ultrasound probe 16a such that the outer shape of the image of the ultrasound probe 16a in the endoscopic image exactly matches the guide 54. The position and the orientation of the ultrasound probe 16a indicated by the guide 54 can be obtained by converting the position and orientation information indicating the position and the orientation of the target ultrasound probe 16a in the robot coordinate system, which is calculated as described above, into the position and orientation information in the camera coordinate system by using a reverse vector of the conversion vector from the camera coordinate system to the robot coordinate system, which is calculated as described above.

The difference between the pre-treatment relative relationship and the current relative relationship can be reduced by processing of the probe control support unit 52. As a result, the operator can obtain the ultrasound tomographic images of the same cross section before the treatment on the treatment target T and after the start of the treatment.

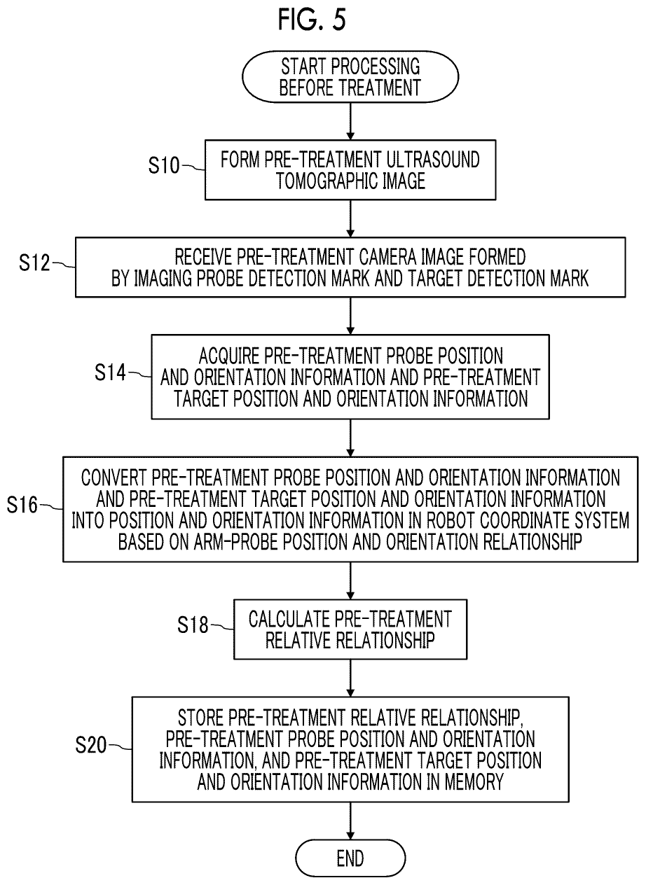

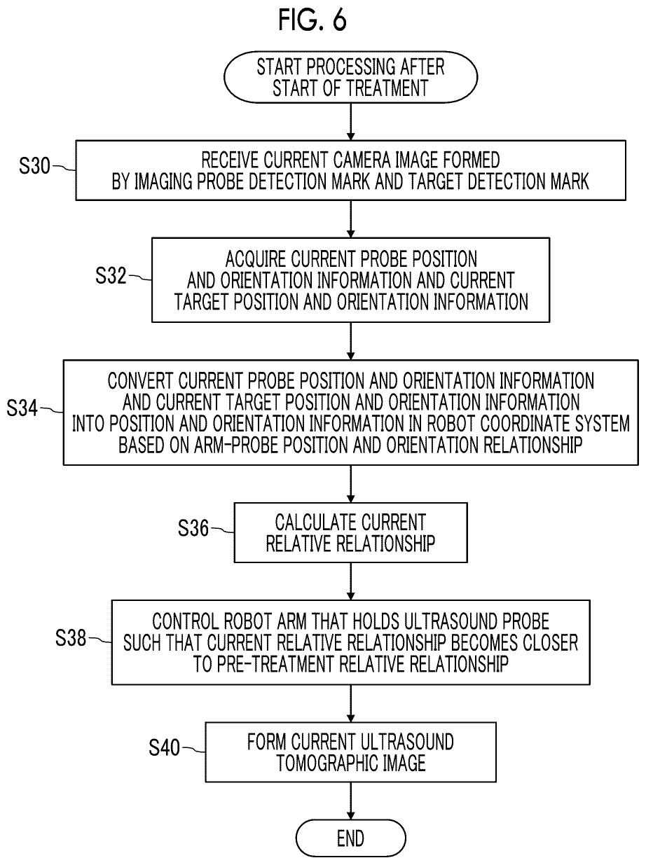

The outline of the configuration of the ultrasound diagnostic apparatus 16 according to the first embodiment has been described above. Hereinafter, a flow of processing of the ultrasound diagnostic apparatus 16 according to the first embodiment will be described with reference to flowcharts shown in FIGS. 5 and 6.

FIG. 5 is a flowchart showing a flow of processing before the treatment on the treatment target T in the first embodiment.

In step S10, the ultrasound probe 16a transmits and receives the ultrasound to and from the treatment target T. The image formation unit 34 forms the pre-treatment ultrasound tomographic image based on the reception signal from the ultrasound probe 16a.

In step S12, the endoscope 14 images the probe detection mark 20 and the target detection mark 22 at the pre-treatment ultrasound transmission and reception time to form the pre-treatment camera image. The ultrasound diagnostic apparatus 16 receives the pre-treatment camera image from the endoscope 14.

In step S14, the pre-treatment relative relationship calculation unit 48 acquires the pre-treatment probe position and orientation information and the pre-treatment target position and orientation information based on the pre-treatment camera image received in step S12.

In step S16, the pre-treatment relative relationship calculation unit 48 converts the pre-treatment probe position and orientation information and the pre-treatment target position and orientation information in the camera coordinate system, which are acquired in step S14, into the pre-treatment probe position and orientation information and the pre-treatment target position and orientation information in the robot coordinate system, based on the arm-probe position and orientation relationship.

In step S18, the pre-treatment relative relationship calculation unit 48 calculates the pre-treatment relative relationship based on the pre-treatment probe position and orientation information and the pre-treatment target position and orientation information in the robot coordinate system, which are acquired in step S16.

In step S20, the pre-treatment relative relationship calculation unit 48 stores the pre-treatment relative relationship calculated in step S18 and the pre-treatment probe position and orientation information and the pre-treatment target position and orientation information in the robot coordinate system, which are acquired in step S16, in the memory 44.

FIG. 6 is a flowchart showing a flow of processing after the start of the treatment on the treatment target T in the first embodiment.

In step S30, the endoscope 14 images the probe detection mark 20 and the target detection mark 22 at the current time to form the current camera image. The ultrasound diagnostic apparatus 16 receives the current camera image from the endoscope 14.

In step S32, the current relative relationship calculation unit 50 acquires the current probe position and orientation information and the current target position and orientation information based on the current camera image received in step S30.

In step S34, the current relative relationship calculation unit 50 converts the current probe position and orientation information and the current target position and orientation information in the camera coordinate system, which are acquired in step S32, into the current probe position and orientation information and the current target position and orientation information in the robot coordinate system, based on the arm-probe position and orientation relationship.

In step S36, the current relative relationship calculation unit 50 calculates the current relative relationship based on the current probe position and orientation information and the current target position and orientation information in the robot coordinate system, which are acquired in step S34.

In step S38, the probe control support unit 52 executes control of bringing the current relative relationship calculated by the current relative relationship calculation unit 50 in step S36 closer to the pre-treatment relative relationship calculated by the pre-treatment relative relationship calculation unit 48 in step S18.

In step S40, the ultrasound probe 16a of which the position and the orientation are changed such that the current relative relationship becomes the pre-treatment relative relationship transmits and receives the ultrasound to and from the treatment target T. The image formation unit 34 forms the current ultrasound tomographic image based on the reception signal from the ultrasound probe 16a. The current ultrasound tomographic image has the same cross section as the pre-treatment ultrasound tomographic image.

### Second Embodiment

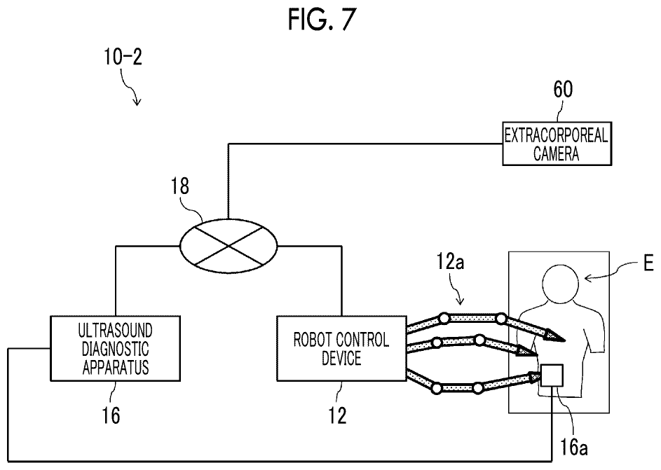

FIG. 7 is a schematic configuration diagram of an operation support system 10-2 for an ultrasound probe according to a second embodiment. The operation support system 10-2 for the ultrasound probe is different from the operation support system 10 for the ultrasound probe according to the first embodiment in that an extracorporeal camera 60 is provided instead of the endoscope 14. Since the points are the same as those in the first embodiment, the duplicated description thereof will be omitted.

The extracorporeal camera 60 includes, in addition to a lens and an image sensor, a processor composed of a CPU and the like, a communication interface composed of a network adapter and the like, and the like. The extracorporeal camera 60 is a camera that is located away from the body surface of the subject E and images the subject E from the outside thereof.

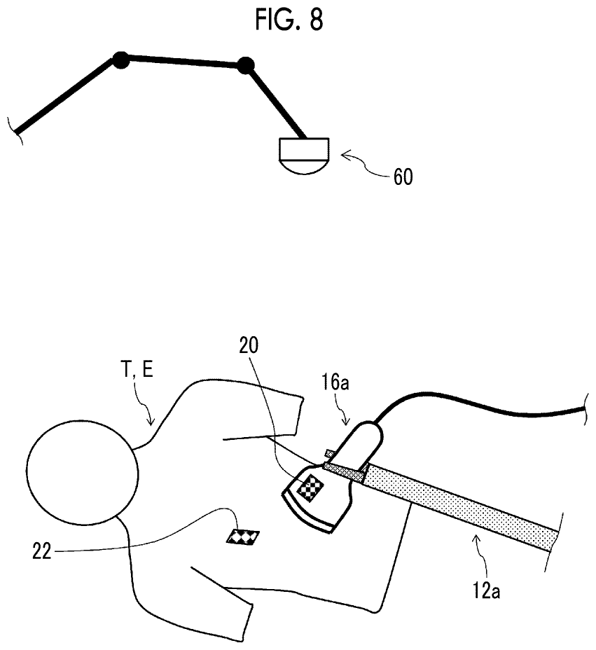

FIG. 8 is a conceptual diagram showing the extracorporeal camera 60, the ultrasound probe 16a, and the subject E having the treatment target T. The extracorporeal camera 60 is located above the subject E by a mechanism such as a camera arm. The extracorporeal camera 60 may be fixed, or a position and an orientation of the extracorporeal camera 60 may be changeable by the operator or the like. In the second embodiment, the probe detection mark 20 is also attached to the ultrasound probe 16a. Further, in the second embodiment, the target detection mark 22 is attached to the treatment target T. In the second embodiment, the target detection mark 22 may be attached to the body surface of the subject E having the treatment target T.

In the second embodiment, the pre-treatment camera image, the current camera image, and the guide screen of the ultrasound probe 16a displayed on the display 38 by the probe control support unit 52 are captured by the extracorporeal camera 60.

### Third Embodiment

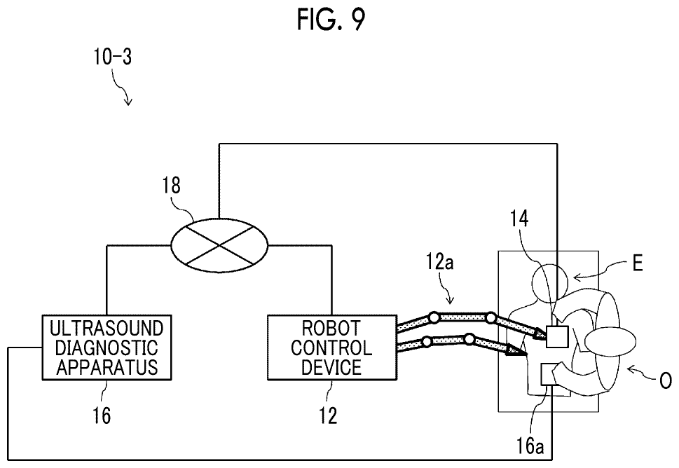

FIG. 9 is a schematic configuration diagram of an operation support system 10-3 for an ultrasound probe according to a third embodiment. In the first embodiment, the robot arm 12a holds the ultrasound probe 16a, but an operator O holds the ultrasound probe 16a in the third embodiment. In addition, in the third embodiment, the endoscope 14 is used as the camera, as in the first embodiment. Since the points are the same as those in the first embodiment, the duplicated description thereof will be omitted.

In the first embodiment, the pre-treatment relative relationship calculation unit 48 and the current relative relationship calculation unit 50 convert the position and orientation information of the ultrasound probe 16a and the treatment target T in the camera coordinate system into the position and orientation information in the robot coordinate system, in accordance with the arm-probe position and orientation relationship that is a fixed position and orientation relationship between the ultrasound probe 16a and the robot arm 12a that holds the ultrasound probe 16a. However, in the third embodiment, since the operator O holds the ultrasound probe 16a, the position and orientation information cannot be converted from the camera coordinate system to the robot coordinate system in accordance with the arm-probe position and orientation relationship.

In the third embodiment, since the endoscope 14 is held by the robot arm 12a, the position and orientation relationship between the endoscope 14 (specifically, the lens thereof) and the robot arm 12a is fixed (not changed). In the present specification, a fixed position and orientation relationship between the endoscope 14 and the robot arm 12a will be referred to as an arm-camera position and orientation relationship. The arm-camera position and orientation relationship can be represented by, for example, a vector in the robot coordinate system.

In the third embodiment, the pre-treatment relative relationship calculation unit 48 converts, in accordance with the arm-camera position and orientation relationship, the position and orientation information of the ultrasound probe 16a, in the camera coordinate system, specified based on the image of the probe detection mark 20 included in the pre-treatment camera image, into the position and orientation information in the robot coordinate system.

Specifically, the pre-treatment relative relationship calculation unit 48 acquires the position and orientation information of the robot arm 12a that holds the endoscope 14 in the robot coordinate system and the information indicating the arm-camera position and orientation relationship from the robot control device 12. The information indicating the arm-camera position and orientation relationship may be input to the robot control device 12 by, for example, the operator who causes the robot arm 12a to hold the endoscope 14. The pre-treatment relative relationship calculation unit 48 can obtain the position and the orientation of the endoscope 14 in the robot coordinate system by using the position and orientation information of the robot arm 12a in the robot coordinate system and the arm-camera position and orientation relationship. That is, the pre-treatment relative relationship calculation unit 48 can obtain an origin of the camera coordinates in the robot coordinate system and a direction (that is, three axial directions of the camera coordinates) of the endoscope 14 (lens thereof). The pre-treatment relative relationship calculation unit 48 can obtain a conversion vector from the camera coordinate system to the robot coordinate system based on the origin of the camera coordinates and three axial directions of the camera coordinates in the robot coordinate system.

The pre-treatment relative relationship calculation unit 48 converts the position and orientation information of the ultrasound probe 16a in the camera coordinate system into the position and orientation information in the robot coordinate system by using the conversion vector.

In addition, the pre-treatment relative relationship calculation unit 48 converts, in accordance with the arm-probe position and orientation relationship, the position and orientation information of the treatment target T, in the camera coordinate system, specified based on the image of the target detection mark 22 included in the pre-treatment camera image into the position and orientation information in the robot coordinate system. Specifically, the pre-treatment relative relationship calculation unit 48 converts the position and orientation information of the treatment target T in the camera coordinate system into the position and orientation information in the robot coordinate system by using the conversion vector.

Similarly, the current relative relationship calculation unit 50 converts, in accordance with the arm-camera position and orientation relationship, the position and orientation information of the ultrasound probe 16a, in the camera coordinate system, specified based on the image of the probe detection mark 20 included in the current camera image into the position and orientation information in the robot coordinate system. Specifically, the current relative relationship calculation unit 50 converts the position and orientation information of the ultrasound probe 16a in the camera coordinate system into the position and orientation information in the robot coordinate system by using the conversion vector.

Furthermore, the current relative relationship calculation unit 50 converts, in accordance with the arm-camera position and orientation relationship, the position and orientation information of the treatment target T, in the camera coordinate system, specified based on the image of the target detection mark 22 included in the current camera image into the position and orientation information in the robot coordinate system. Specifically, the current relative relationship calculation unit 50 converts the position and orientation information of the treatment target T in the camera coordinate system into the position and orientation information in the robot coordinate system by using the conversion vector.

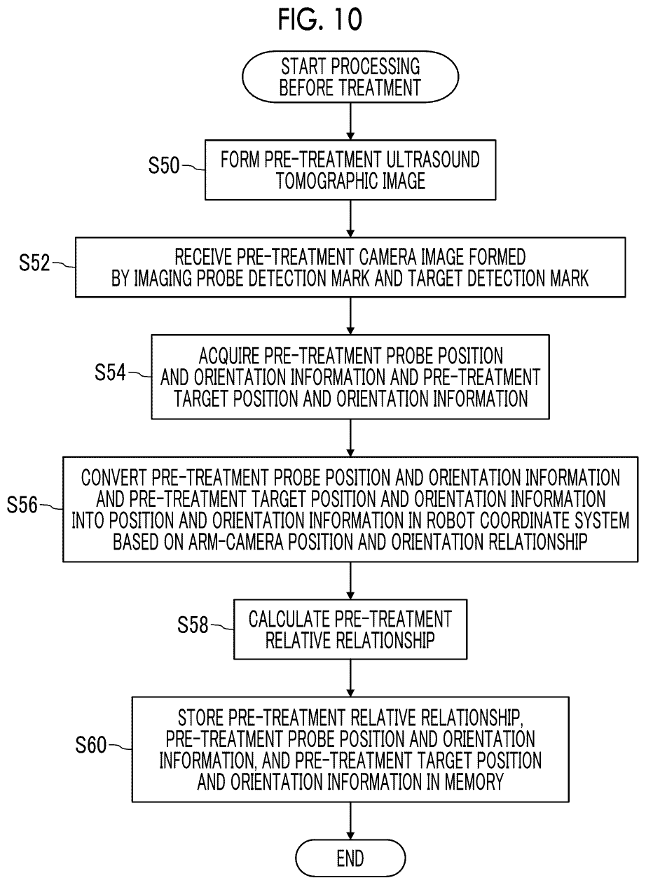

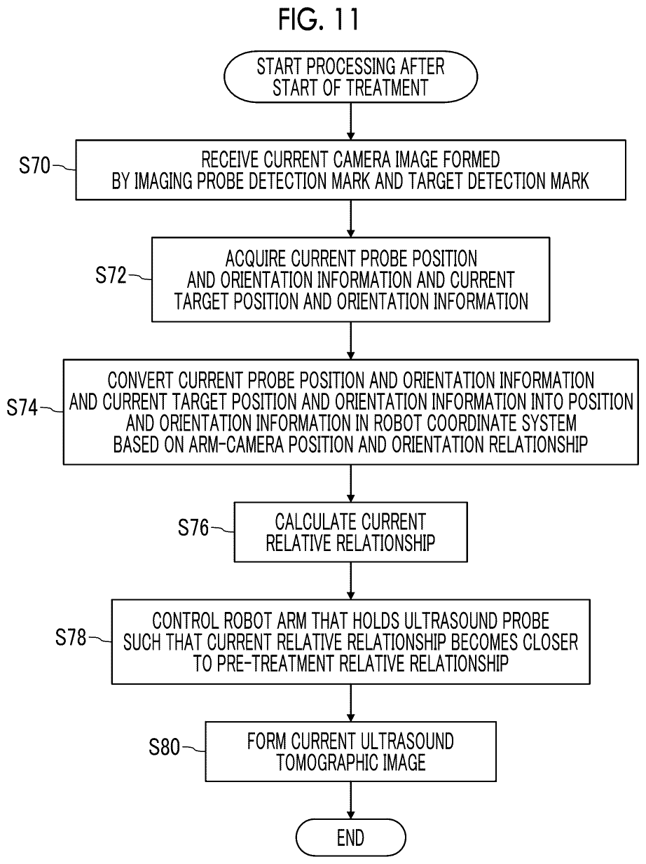

Hereinafter, a flow of processing of an ultrasound diagnostic apparatus 16-3 according to the third embodiment will be described with reference to flowcharts shown in FIGS. 10 and 11.

FIG. 10 is a flowchart showing a flow of processing before the treatment on the treatment target T in the third embodiment.

In step S50, the ultrasound probe 16a transmits and receives the ultrasound to and from the treatment target T. The image formation unit 34 forms the pre-treatment ultrasound tomographic image based on the reception signal from the ultrasound probe 16a.

In step S52, the endoscope 14 images the probe detection mark 20 and the target detection mark 22 at the pre-treatment ultrasound transmission and reception time to form the pre-treatment camera image. The ultrasound diagnostic apparatus 16 receives the pre-treatment camera image from the endoscope 14.

In step S54, the pre-treatment relative relationship calculation unit 48 acquires the pre-treatment probe position and orientation information and the pre-treatment target position and orientation information based on the pre-treatment camera image received in step S52.

In step S56, the pre-treatment relative relationship calculation unit 48 converts the pre-treatment probe position and orientation information and the pre-treatment target position and orientation information in the camera coordinate system, which are acquired in step S54, into the pre-treatment probe position and orientation information and the pre-treatment target position and orientation information in the robot coordinate system, based on the arm-camera position and orientation relationship.

In step S58, the pre-treatment relative relationship calculation unit 48 calculates the pre-treatment relative relationship based on the pre-treatment probe position and orientation information and the pre-treatment target position and orientation information in the robot coordinate system, which are acquired in step S56.

In step S60, the pre-treatment relative relationship calculation unit 48 stores the pre-treatment relative relationship calculated in step S58 and the pre-treatment probe position and orientation information and the pre-treatment target position and orientation information in the robot coordinate system, which are acquired in step S56, in the memory 44.

FIG. 11 is a flowchart showing a flow of processing after the start of the treatment on the treatment target T.

In step S70, the endoscope 14 images the probe detection mark 20 and the target detection mark 22 at the current time to form the current camera image. The ultrasound diagnostic apparatus 16 receives the current camera image from the endoscope 14.

In step S72, the current relative relationship calculation unit 50 acquires the current probe position and orientation information and the current target position and orientation information based on the current camera image received in step S70.

In step S74, the current relative relationship calculation unit 50 converts the current probe position and orientation information and the current target position and orientation information in the camera coordinate system, which are acquired in step S72, into the current probe position and orientation information and the current target position and orientation information in the robot coordinate system, based on the arm-camera position and orientation relationship.

In step S76, the current relative relationship calculation unit 50 calculates the current relative relationship based on the current probe position and orientation information and the current target position and orientation information in the robot coordinate system, which are acquired in step S74.

In step S78, the probe control support unit 52 executes control of bringing the current relative relationship calculated by the current relative relationship calculation unit 50 in step S76 closer to the pre-treatment relative relationship calculated by the pre-treatment relative relationship calculation unit 48 in step S58.

In step S80, the ultrasound probe 16a of which the position and the orientation are changed such that the current relative relationship becomes the pre-treatment relative relationship transmits and receives the ultrasound to and from the treatment target T. The image formation unit 34 forms the current ultrasound tomographic image based on the reception signal from the ultrasound probe 16a. The current ultrasound tomographic image has the same cross section as the pre-treatment ultrasound tomographic image.

### Fourth Embodiment

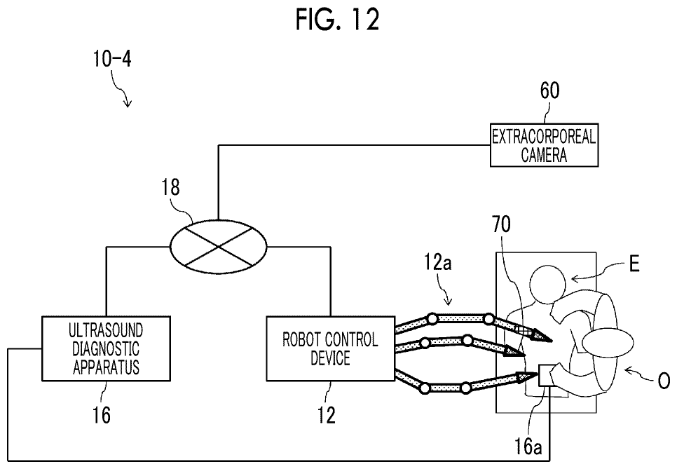

FIG. 12 is a schematic configuration diagram of an operation support system 10-4 for an ultrasound probe according to a fourth embodiment. The operation support system 10-4 for the ultrasound probe is different from the operation support system 10 for the ultrasound probe according to the first embodiment in that the extracorporeal camera 60 is provided instead of the endoscope 14, and the operator O holds the ultrasound probe 16a. Since the points are the same as those in the first embodiment, the duplicated description thereof will be omitted.

In the fourth embodiment, as in the third embodiment, the operator O holds the ultrasound probe 16a, and thus the position and orientation information cannot be converted from the camera coordinate system to the robot coordinate system in accordance with the arm-probe position and orientation relationship. In addition, in the fourth embodiment, since the extracorporeal camera 60 is used instead of the endoscope 14, the position and orientation information cannot be converted from the camera coordinate system to the robot coordinate system even in accordance with the arm-camera position and orientation relationship as in the third embodiment.

Therefore, in the fourth embodiment, an arm detection mark 70 is attached to at least one of the robot arms 12a. The arm detection mark 70 is a mark having a pattern indicating the position and the orientation of the robot arm 12a. Accordingly, the arm detection mark 70 is imaged by the extracorporeal camera 60 to acquire an extracorporeal camera image, and the image of the arm detection mark 70 included in the extracorporeal camera image is analyzed to detect the position and the orientation of the robot arm 12a. Examples of the arm detection mark 70 include an AR marker.

The extracorporeal camera 60 images the probe detection mark 20 attached to the ultrasound probe 16a, the target detection mark 22 attached to the treatment target T (for example, the body surface of the subject E), and the arm detection mark 70 to form the extracorporeal camera image.

In the fourth embodiment, the pre-treatment relative relationship calculation unit 48 specifies the position and the orientation of the robot arm 12a in the camera coordinate system based on the image of the arm detection mark 70 included in the pre-treatment camera image formed by the extracorporeal camera 60. Then, the pre-treatment relative relationship calculation unit 48 acquires an inter-coordinate system relationship indicating a relationship between the camera coordinates and the robot coordinates based on the specified position and orientation of the robot arm 12a in the camera coordinate system and the position and the orientation of the robot arm 12a in the robot coordinate system. As necessary, the robot arm 12a may be moved to acquire a plurality of combinations of the position and the orientation of the robot arm 12a in the camera coordinate system and the position and the orientation of the robot arm 12a in the robot coordinate system, and the inter-coordinate system relationship may be acquired based on the plurality of combinations. The inter-coordinate system relationship is the conversion vector from the camera coordinate system to the robot coordinate system.

The pre-treatment relative relationship calculation unit 48 converts, in accordance with the inter-coordinate system relationship acquired as described above, the position and orientation information of the ultrasound probe 16a, in the camera coordinate system, specified based on the image of the probe detection mark 20 included in the pre-treatment camera image into the position and orientation information in the robot coordinate system.

In addition, the pre-treatment relative relationship calculation unit 48 converts, in accordance with the inter-coordinate system relationship, the position and orientation information of the treatment target T, in the camera coordinate system, specified based on the image of the target detection mark 22 included in the pre-treatment camera image into the position and orientation information in the robot coordinate system.

Similarly, the current relative relationship calculation unit 50 converts, in accordance with the inter-coordinate system relationship, the position and orientation information of the ultrasound probe 16a, in the camera coordinate system, specified based on the image of the probe detection mark 20 included in the current camera image into the position and orientation information in the robot coordinate system.

Further, the current relative relationship calculation unit 50 converts, in accordance with the inter-coordinate system relationship, the position and orientation information of the treatment target T, in the camera coordinate system, specified based on the image of the target detection mark 22 included in the current camera image into the position and orientation information in the robot coordinate system.

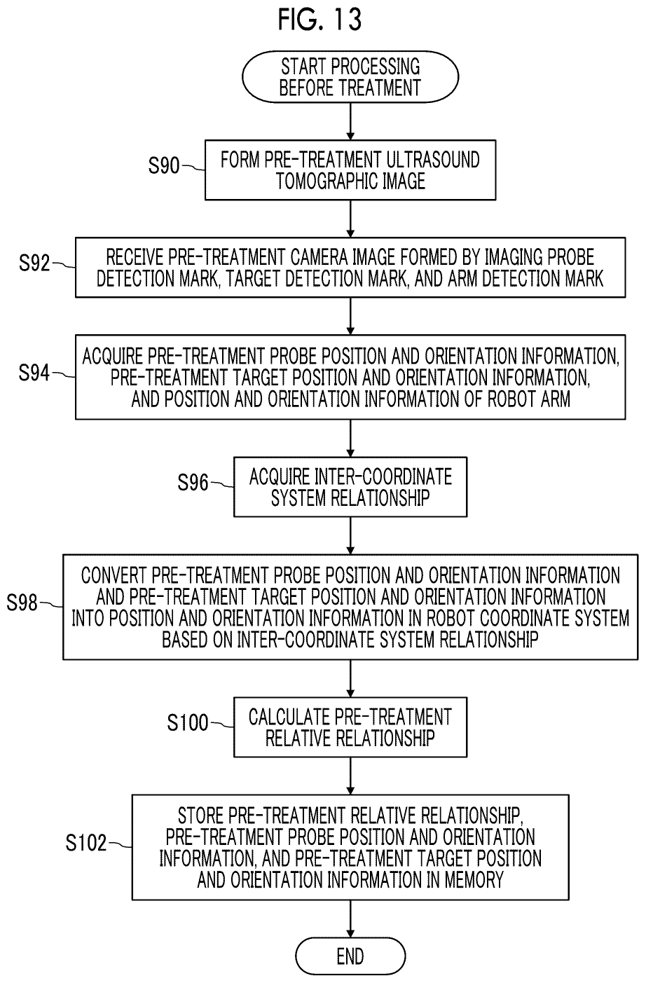

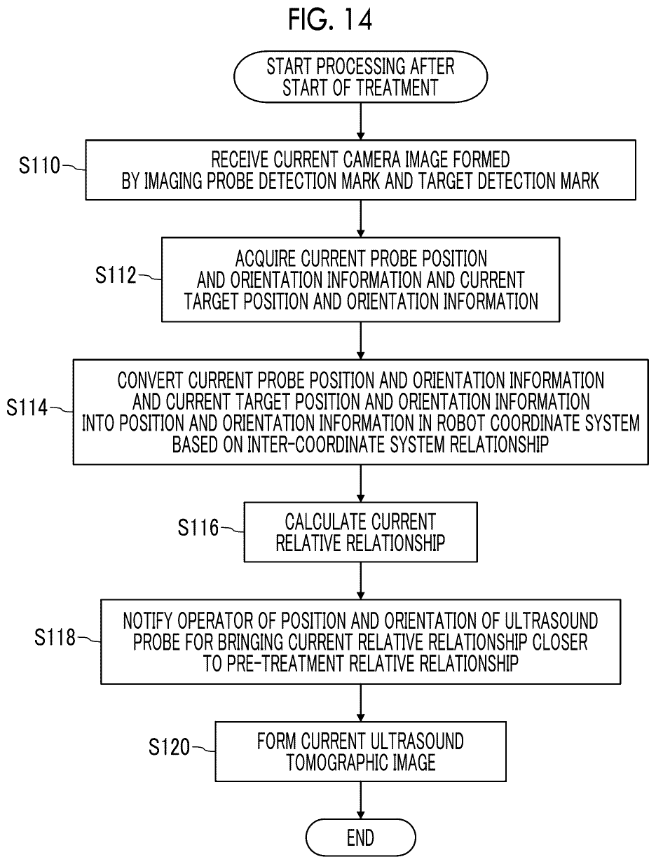

Hereinafter, a flow of processing of the ultrasound diagnostic apparatus 16 according to the fourth embodiment will be described with reference to flowcharts shown in FIGS. 13 and 14.

FIG. 13 is a flowchart showing a flow of processing before the treatment on the treatment target T in the fourth embodiment.

In step S90, the ultrasound probe 16a transmits and receives the ultrasound to and from the treatment target T. The image formation unit 34 forms the pre-treatment ultrasound tomographic image based on the reception signal from the ultrasound probe 16a.

In step S92, the extracorporeal camera 60 images the probe detection mark 20, the target detection mark 22, and the arm detection mark 70 at the pre-treatment ultrasound transmission and reception time to form the pre-treatment camera image. The ultrasound diagnostic apparatus 16 receives the pre-treatment camera image from the extracorporeal camera 60.

In step S94, the pre-treatment relative relationship calculation unit 48 acquires the pre-treatment probe position and orientation information, the pre-treatment target position and orientation information, and the position and orientation information of the robot arm 12a based on the pre-treatment camera image received in step S92.

In step S96, the pre-treatment relative relationship calculation unit 48 acquires the inter-coordinate system relationship based on the position and the orientation (pre-treatment probe position and orientation information) of the robot arm 12a, in the camera coordinate system, specified in step S94 and the position and the orientation of the robot arm 12a in the robot coordinate system.

In step S98, the pre-treatment relative relationship calculation unit 48 converts the pre-treatment probe position and orientation information and the pre-treatment target position and orientation information in the camera coordinate system, which are acquired in step S94, into the pre-treatment probe position and orientation information and the pre-treatment target position and orientation information in the robot coordinate system, based on the inter-coordinate system relationship acquired in step S96.

In step S100, the pre-treatment relative relationship calculation unit 48 calculates the pre-treatment relative relationship based on the pre-treatment probe position and orientation information and the pre-treatment target position and orientation information in the robot coordinate system, which are acquired in step S98.

In step S102, the pre-treatment relative relationship calculation unit 48 stores the pre-treatment relative relationship calculated in step S100 and the pre-treatment probe position and orientation information and the pre-treatment target position and orientation information in the robot coordinate system, which are acquired in step S98 in the memory 44.

FIG. 14 is a flowchart showing a flow of processing after the start of the treatment on the treatment target T in the fourth embodiment.

In step S110, the extracorporeal camera 60 images the probe detection mark 20 and the target detection mark 22 at the current time to form the current camera image. The ultrasound diagnostic apparatus 16 receives the current camera image from the extracorporeal camera 60.

In step S112, the current relative relationship calculation unit 50 acquires the current probe position and orientation information and the current target position and orientation information based on the current camera image received in step S30.

In step S114, the current relative relationship calculation unit 50 converts the current probe position and orientation information and the current target position and orientation information in the camera coordinate system, which are acquired in step S112, into the current probe position and orientation information and the current target position and orientation information in the robot coordinate system, based on the inter-coordinate system relationship acquired in step S96.

In step S116, the current relative relationship calculation unit 50 calculates the current relative relationship based on the current probe position and orientation information and the current target position and orientation information in the robot coordinate system, which are acquired in step S114.

In step S118, the probe control support unit 52 executes control of bringing the current relative relationship calculated by the current relative relationship calculation unit 50 in step S116 closer to the pre-treatment relative relationship calculated by the pre-treatment relative relationship calculation unit 48 in step S100.

In step S120, the ultrasound probe 16a of which the position and the orientation are changed such that the current relative relationship becomes the pre-treatment relative relationship transmits and receives the ultrasound to and from the treatment target T. The image formation unit 34 forms the current ultrasound tomographic image based on the reception signal from the ultrasound probe 16a. The current ultrasound tomographic image has the same cross section as the pre-treatment ultrasound tomographic image.

Although the operation support system for the ultrasound probe according to the present disclosure has been described above, the operation support system for the ultrasound probe according to the present disclosure is not limited to the above-described embodiments, and various changes can be made without departing from the gist of the present disclosure.

For example, in each of the above-described embodiments, the operation support systems 10, 10-2, 10-3, and 10-4 for the ultrasound probe perform control of making the pre-treatment ultrasound tomographic image and the current ultrasound tomographic image represent the same cross section, but the operation support systems 10, 10-2, 10-3, and 10-4 for the ultrasound probe can be used for other purposes as long as the relative position and orientation of the ultrasound probe 16a with respect to the treatment target T are the same between before the treatment on the treatment target T and after the start of the treatment. For example, even in a case in which the ultrasound probe 16a transmits and receives the ultrasound to and from the treatment target T with the same position and orientation before the treatment and after the start of the treatment, to form the color Doppler images or to perform various measurements before the treatment and after the start of the treatment, the operation support systems 10, 10-2, 10-3, and 10-4 for the ultrasound probe can be used.

In addition, in each of the above-described embodiments, the position and orientation information of the ultrasound probe 16a and the position and orientation information of the treatment target T in the camera coordinate system are converted into the position and orientation information in the robot coordinate system, but, in a case in which the position and the orientation of the endoscope 14 or the extracorporeal camera 60 do not change before the treatment and after the start of the treatment (that is, at the current time), it is not necessary to perform the conversion from the camera coordinate system to the robot coordinate system. In this case, the pre-treatment relative relationship calculation unit 48 may calculate the pre-treatment relative relationship based on the position and orientation information of the ultrasound probe 16a and the position and orientation information of the treatment target T in the camera coordinate system, and the current relative relationship calculation unit 50 may calculate the current relative relationship based on the position and orientation information of the ultrasound probe 16a and the position and orientation information of the treatment target T in the camera coordinate system.

In addition, in each of the above-described embodiments, the controller 46 of the ultrasound diagnostic apparatus 16 has each of the functions of the pre-treatment relative relationship calculation unit 48, the current relative relationship calculation unit 50, and the probe control support unit 52, but these functions need not always be performed by the ultrasound diagnostic apparatus 16. For example, these functions may be exhibited by a server computer or the like that is connected to the robot control device 12, the endoscope 14 (or the extracorporeal camera 60), and the ultrasound diagnostic apparatus 16 so as to be able to communicate with each other. At least one of the above-described functions may be exhibited by the robot control device 12. Further, all of the above-described functions may be exhibited by cooperation of a plurality of devices without being exhibited by one device.

What is claimed is:

1. An operation support system for an ultrasound probe, comprising:
a pre-treatment relative relationship calculation unit that calculates, based on a pre-treatment camera image obtained by imaging a probe detection mark attached to an ultrasound probe and a target detection mark attached to a treatment target with a camera before a treatment on a subject, a pre-treatment relative relationship that is a relative position and orientation relationship between the ultrasound probe and the treatment target in a case in which ultrasound is transmitted to and received from the subject for forming an ultrasound image before the treatment on the subject;
a current relative relationship calculation unit that calculates, based on a current camera image obtained by imaging the probe detection mark and the target detection mark with the camera at a current time after a start of the treatment on the subject, a current relative relationship that is a relative position and orientation relationship between the ultrasound probe and the treatment target at the current time; and
a probe control support unit that executes control of bringing the current relative relationship closer to the pre-treatment relative relationship.

2. The operation support system for an ultrasound probe according to claim 1,
wherein the probe control support unit brings the current relative relationship closer to the pre-treatment relative relationship by changing a position or an orientation of a robot arm that holds the ultrasound probe.

3. The operation support system for an ultrasound probe according to claim 1,
wherein the probe control support unit notifies an operator who operates the ultrasound probe of a position and an orientation of the ultrasound probe for achieving the current relative relationship.

4. The operation support system for an ultrasound probe according to claim 3,
wherein the probe control support unit displays, on a display unit, a camera image that is obtained by imaging the ultrasound probe and the treatment target with the camera and that shows the position and the orientation of the ultrasound probe for achieving the current relative relationship.

5. The operation support system for an ultrasound probe according to claim 1,
wherein the pre-treatment relative relationship calculation unit
converts, in accordance with an arm-probe position and orientation relationship that is a fixed position and orientation relationship between a robot arm and the ultrasound probe held by the robot arm, position and orientation information indicating a position and an orientation of the ultrasound probe, in a camera coordinate system, specified based on an image of the probe detection mark included in the pre-treatment camera image into position and orientation information, in a robot coordinate system, recognized by a robot control device that controls the robot arm,
converts, in accordance with the arm-probe position and orientation relationship, position and orientation information indicating a position and an orientation of the treatment target, in the camera coordinate system, specified based on an image of the target detection mark included in the pre-treatment camera image into position and orientation information in the robot coordinate system, and
calculates the pre-treatment relative relationship in the robot coordinate system, and
the current relative relationship calculation unit
converts, in accordance with the arm-probe position and orientation relationship, position and orientation information indicating a position and an orientation of the ultrasound probe, in the camera coordinate system, specified based on an image of the probe detection mark included in the current camera image into position and orientation information in the robot coordinate system,
converts, in accordance with the arm-probe position and orientation relationship, position and orientation information indicating a position and an orientation of the treatment target, in the camera coordinate system, specified based on an image of the target detection mark included in the current camera image into position and orientation information in the robot coordinate system, and
calculates the current relative relationship in the robot coordinate system.

6. The operation support system for an ultrasound probe according to claim 1,
wherein the pre-treatment relative relationship calculation unit
converts, in accordance with an arm-camera position and orientation relationship that is a fixed position and orientation relationship between a robot arm and the camera held by the robot arm, position and orientation information indicating a position and an orientation of the ultrasound probe, in a camera coordinate system, specified based on an image of the probe detection mark included in the pre-treatment camera image into position and orientation information, in a robot coordinate system, recognized by a robot control device that controls the robot arm,
converts, in accordance with the arm-camera position and orientation relationship, position and orientation information indicating a position and an orientation of the treatment target, in the camera coordinate system, specified based on an image of the target detection mark included in the pre-treatment camera image into position and orientation information in the robot coordinate system, and
calculates the pre-treatment relative relationship in the robot coordinate system, and
the current relative relationship calculation unit
converts, in accordance with the arm-camera position and orientation relationship, position and orientation information indicating a position and an orientation of the ultrasound probe, in the camera coordinate system, specified based on an image of the probe detection mark included in the current camera image into position and orientation information in the robot coordinate system,
converts, in accordance with the arm-camera position and orientation relationship, position and orientation information indicating a position and an orientation of the treatment target, in the camera coordinate system, specified based on an image of the target detection mark included in the current camera image into position and orientation information in the robot coordinate system, and
calculates the current relative relationship in the robot coordinate system.

7. The operation support system for an ultrasound probe according to claim 1,
wherein the pre-treatment camera image and the current camera image are obtained by imaging the probe detection mark, the target detection mark, and an arm detection mark attached to a robot arm with the camera,
the pre-treatment relative relationship calculation unit
converts, in accordance with an inter-coordinate system relationship indicating a relationship between camera coordinates and robot coordinates and obtained based on a position and an orientation of the robot arm, in a camera coordinate system, specified based on an image of the arm detection mark included in the pre-treatment camera image and a position and an orientation of the robot arm, in a robot coordinate system, recognized by a robot control device that controls the robot arm, position and orientation information indicating a position and an orientation of the ultrasound probe, in the camera coordinate system, specified based on an image of the probe detection mark included in the pre-treatment camera image into position and orientation information in the robot coordinate system,
converts, in accordance with the inter-coordinate system relationship, position and orientation information indicating a position and an orientation of the treatment target, in the camera coordinate system, specified based on an image of the target detection mark included in the pre-treatment camera image into position and orientation information in the robot coordinate system, and
calculates the pre-treatment relative relationship in the robot coordinate system, and
the current relative relationship calculation unit
converts, in accordance with the inter-coordinate system relationship, position and orientation information indicating a position and an orientation of the ultrasound probe, in the camera coordinate system, specified based on an image of the probe detection mark included in the current camera image into position and orientation information in the robot coordinate system,
converts, in accordance with the inter-coordinate system relationship, position and orientation information indicating a position and an orientation of the treatment target, in the camera coordinate system, specified based on an image of the target detection mark included in the current camera image into position and orientation information in the robot coordinate system, and
calculates the current relative relationship in the robot coordinate system.

8. A non-transitory computer-readable storage medium storing an operation support program for an ultrasound probe, causing a computer to function as:
a pre-treatment relative relationship calculation unit that calculates, based on a pre-treatment camera image obtained by imaging a probe detection mark attached to an ultrasound probe and a target detection mark attached to a treatment target with a camera before a treatment on a subject, a pre-treatment relative relationship that is a relative position and orientation relationship between the ultrasound probe and the treatment target in a case in which ultrasound is transmitted to and received from the subject for forming an ultrasound image before the treatment on the subject;
a current relative relationship calculation unit that calculates, based on a current camera image obtained by imaging the probe detection mark and the target detection mark with the camera at a current time after a start of the treatment on the subject, a current relative relationship that is a relative position and orientation relationship between the ultrasound probe and the treatment target at the current time; and
a probe control support unit that executes control of bringing the current relative relationship closer to the pre-treatment relative relationship.
